(19) United States
(12) Patent Application Publication
(10) Pub. No.: US 2010/0284546 A1
(43) Pub. Date: Nov. 11, 2010

(54) Active noise control algorithm that requires no secondary path identification based on the SPR property

(76) Inventors: Victor DeBrunner, Tallahassee, FL (US); Dayong Zhou, Norman, OK (US); Linda DeBrunner, Tallahassee, FL (US); Justin Fuller, Norman, OK (US); Yunhua Wang, Norman, OK (US)

Correspondence Address: DUNLAP CODDING, P.C., PO BOX 16370, OKLAHOMA CITY, OK, 73113, US

(21) Appl. No.: 11/506,256

(22) Filed: Aug. 18, 2006

Related U.S. Application Data
(60) Provisional application No. 60/709,324, filed on Aug. 18, 2005.

(51) Int. Cl. A61F 11/06

(52) U.S. Cl. 381/71.2

(57) ABSTRACT

A control system for reducing noise or vibration in a target zone. The noise or vibration is produced by a source and transferred to the target zone by a main path. The control system is provided with an actuator, at least one error sensor and a controller. The actuator is positioned to deliver actuated signals into at least a portion of the target zone. The at least one error sensor monitors the residual noise or vibration power in the target zone and produces an error signal representative thereof. The controller receives a reference signal representative of noise or vibration produced by the source, and the error signal representative of the residual noise power in the target zone. The controller analyzes sub-bands of the reference signal and the error signal without identification of a secondary path, and provides drive signals to the actuator to cause the actuator to deliver the actuated signals into the target zone so as to reduce the residual noise power in the target zone.

### CROSS-REFERENCE TO RELATED APPLICATIONS

The present patent application claims priority to the provisional patent application identified by U.S. Ser. No. 60/709,324, filed on Aug. 18, 2005, the entire content of which is hereby incorporated herein by reference.

### STATEMENT REGARDING FEDERALLY SPONSORED RESEARCH OR DEVELOPMENT

The research for the present invention was supported, at least in part, by DOT/Federal Highway Administration Contract No. DTFH61-01-X-00050.

### THE NAMES OF THE PARTIES TO A JOINT RESEARCH AGREEMENT

Not Applicable.

### REFERENCE TO A “SEQUENCE LISTING,” A TABLE, OR A COMPUTER PROGRAM LISTING APPENDIX SUBMITTED ON A COMPACT DISC AND AN INCORPORATION-BY-REFERENCE OF THE MATERIAL ON THE COMPACT DISC

Not Applicable.

### BACKGROUND OF THE INVENTION

Active noise control (ANC) and active vibration control (AVC) has received much attention in the recent research literature and for industrial applications. Based on the superposition principle, the undesired noise or vibration can be reduced by adding another noise or vibration with the same amplitude but opposite sign, which is generated by actuators such as loudspeakers in ANC or piezoelectric materials in AVC [1], [2]. The filtered-x LMS algorithm is the most common algorithm applied in both feed-forward and feedback ANC due to its ease of implementation.

Most available active noise control algorithms, including the filtered-x LMS algorithm, require identification of the secondary path, which is defined as the path leading from the adaptive filter output to the error sensor that measures the residual noise. Thus, the secondary path includes the D/A converter, power amplifier, actuator, physical path, error sensor, and other components. The requirement of identifying the secondary path causes several problems to the control system: 1) it increases the complexity of the control system implementation; 2) errors in identifying the secondary path may cause the adaptive algorithm to diverge, ruining the control system performance; and 3) the online identification often requires an auxiliary noise input that contributes to the residual noise power.

Several researchers have observed these problems and as a result they have developed variations of the filtered-x LMS algorithm that improve the control system performance and robustness while reducing the impact of the auxiliary noise [3]-[7]. However, each of these algorithms increases the control system complexity. A control algorithm that does not require secondary path identification is a ready solution to these problems. Currently, there are several available ANC algorithms that do not require secondary path estimation [8]-[14]. The methods introduced by Feintuch et al [8] and Bjarnason et al [9] require a priori information regarding the secondary path. These methods are constrained—they only work for certain narrowband noises and systems. The algorithm introduced in [12] is based on the simultaneous equation method, and so requires another auxiliary filter to create the noise control filter. Although this technique converges quickly, it also requires a complex system configuration with a greatly increased computational burden. The method introduced in [13], [14] requires three adaptive filters that simultaneously minimize two “artificial” errors. This method also greatly increases the system complexity and computational burden. In [10], [11], random search algorithms based on a simple parameter perturbation optimization method are employed to find the coefficients of the adaptive control filter. Although simple in structure, the proposed methods converge very slowly when compared to efficient adaptive (gradient based) algorithms such as the filtered-x LMS. Furthermore, the added perturbations contribute to the residual noise power.

Here, a new adaptive control algorithm to cancel single-tone noise, narrowband noise, and broadband noise is introduced that does not require any secondary path identification. The proposed method enjoys simple structures, good performance, and reasonable convergence speed. These ideas were initially introduced by the authors in [15].

### 1. A Geometric Analysis of the Filtered-x LMS Algorithm

An example of the filtered-x LMS algorithm is schematically illustrated in FIG. 1. The filtered x LMS algorithm could be applied to both feed-forward ANC (see FIG. 1) and feedback ANC. In FIG. 1, P(z), S(z), and Ŝ(z) represent the main path, secondary path, and estimated secondary path, respectively; W(z) is the adaptive filter; x(n) is the reference signal, and v(n) is an additive zero-mean noise, which is uncorrelated with x(n). Define the reference signal vector x(n)=[x(n) x(n−1) . . . x(n−M)]^{T}, where M is the order of adaptive filter w(n). The adaptive filter coefficients are updated by

w(n)=w(n−1)+μe(n)x_{f}*(n) (1)

where x_{f}(n) is the reference signal vector x(n) filtered by the estimated secondary path: Ŝ(z), and superscript * denotes complex conjugate. The positive, real number μ is the step size, which controls the convergence speed and stability of the adaptive algorithm.

If the input (i.e., the reference signal) is assumed to be a pure sinusoid with frequency ω, then each of the filters P(z), W(z), S(z), and Ŝ(z) can be represented by complex numbers P_{ω}, W_{ω}(n), S_{ω}, and Ŝ_{ω}, respectively, which represent the gain and phase at the frequency ω. Thus, for a single-frequency input, (1) is now

$$\begin{matrix}{W}_{ω} ( n ) = {W}_{ω} ( n - 1 ) + μ {x}_{ω}^{*} ( n ) {{S}^{⋒}}_{ω} [ {x}_{ω} ( n ) {P}_{ω} - {x}_{ω} ( n ) {W}_{ω} ( n - 1 ) {S}_{ω} ] \\ = {W}_{ω} ( n - 1 ) + μ {x}_{*}^{*} ( n ) {x}_{ω} ( n ) {{S}^{⋒}}_{ω} {S}_{ω} [ {P}_{ω} / {S}_{ω} - {W}_{ω} ( n - 1 ) ] \\ = {W}_{ω} ( n - 1 ) + μ {P}_{x} ( ω ) {{S}^{⋒}}_{ω}^{*} {S}_{ω} [ {P}_{ω} / {S}_{ω} - {W}_{ω} ( n - 1 )\end{matrix} \qquad ( 2 )$$

where Pₓ(ω) represents the power of the reference signal at the frequency ω. Note that here the additive noise v(n) is not included since it has zero mean and is uncorrelated with the reference signal x(n). When the adaptive filter converges, W_{ω}(n)=W_{ω}(n−1) and so W_{ω}(∞)=P_{ω}/S_{ω}.

If the estimated secondary path Ŝ(z) has no error, i.e., Ŝ(z)=s(z), then (2) becomes

W_{ω}′(n)=W_{ω}(n−1)+μPₓ(ω)|S_{ω}|²[P_{ω}/S_{ω}−W_{ω}(n−1)]. (3)

The physical meaning of (3) is this: as W_{ω}′(n) goes in a point-to-point direction from W_{ω}(n−1) towards P_{ω}/S_{ω}, the filter travels a length μPₓ(ω)|S_{ω}|²|P_{ω}/S_{ω}−W_{ω}(n−1)| as shown in FIG. 2. μPₓ(ω)|S_{ω}|²<2 ensures the convergence of the adaptive filter. However, in practice, there is always some estimation error. At the frequency ω, the estimated secondary path Ŝ(z) can be expressed as:

Ŝ_{ω}=c_{ω}S_{ω}e^{jθ}^{_{ω}} (4)

where c_{ω} is a real constant representing the amplitude estimation error, and θ_{ω} represents the phase estimation error. Combining (4) and (2) yields

W_{ω}(n)=W_{ω}(n−1)+μPₓ(ω)|S_{ω}|²c_{ω}[P_{ω}/S_{ω}−W_{ω}(n−1)]e^{-jθ}^{_{ω}}. (5)

Consequently, the W_{ω}(n) doesn't go in a point-to-point direction from W_{ω}(n−1) directly towards P_{ω}/S_{ω}; instead there is an angle difference (separation) θ_{ω}, as shown in FIG. 2. As long as this angle satisfies |θ_{ω}|<90° and μc_{ω}Pₓ(ω)|S_{ω}|²<2 cos(θ_{ω}), then the distance from W_{ω}(n) to P_{ω}/S_{ω} will be less than the distance from W_{ω}(n−1) to P_{ω}/S_{ω}. Accordingly, the update W_{ω}(n) is closer to the optimum solution than is W_{ω}(n−1) and so the adaptive filter will still eventually converge. On the other hand, when |θ_{ω}|≧90°, the adaptive filter will never converge, no matter how small the step size is chosen to be.

Although this analysis is based on single-frequency inputs, the result can be extended to broadband input signals using orthogonal filtering. In this case, the step size μ should take on the smallest value over the frequency range, i.e.

$$μ < {min}_{ω} \frac{2 cos ( {θ}_{ω} )}{{c}_{ω} {P}_{x} ( ω ) {\vert {S}_{ω} \vert}^{2}} \qquad ( 6 )$$

This analysis shows the impact of the ±90° stability bound [1] of the filtered-x LMS algorithm, which is equivalent to the strictly positive real (SPR) condition in [8]. The amplitude estimation error of Ŝ(z) will only affect the allowable range for the step size μ—these errors will not cause the adaptive filter to diverge for a correct choice of μ. This situation has been observed by many researchers [8], [16]-[18]. However our analysis provides some geometrical meaning and intuitive explanation of this condition, and we are going to develop our new algorithm based on this analysis and the SPR property.

### BRIEF SUMMARY OF THE INVENTION

In an aspect, the present invention relates to a control system for reducing noise or vibration in a target zone. The noise or vibration is produced by a source and is transferred to the target zone by a main path. The control system includes an actuator, at least one error sensor, and a controller. The actuator delivers actuated signals into at least a portion of the target zone. The error sensor monitors the residual noise or vibration power in the target zone and produces an error signal representative thereof. The controller receives a reference signal representative of noise or vibration produced by the source and the error signal representative of the residual noise power in the target zone. The controller analyzes sub-bands of the reference signal and the error signal without identification of a secondary path, and provides drive signals to the actuator to cause the actuator to deliver the actuated signals into the target zone so as to reduce the residual noise power in the target zone.

In another aspect, the present invention relates to a control algorithm stored on a computer readable medium. The control algorithm includes an algorithm that receives a reference signal indicative of noise produced by a source and an algorithm that receives an error signal representative of the residual noise power in a target zone. The control algorithm also includes another algorithm for analyzing sub-bands of the reference signal and the error signal without identification of a secondary path and an algorithm for providing adaptive filter coefficients to an adaptive filter.

In yet another aspect, the present invention relates to a controller that reduces noise or vibration in a target zone. The noise is produced by a source and transferred to the target zone by a main path. The controller includes a computational system running a control algorithm. The control algorithm causes the computational system to receive a reference signal representative of noise or vibration produced by the source and an error signal representative of the residual noise power in the target zone. The control algorithm causes the computational system to analyze sub-bands of the reference signal and the error signal without identification of a secondary path to update adaptive filter coefficients.

Another aspect of the invention relates to a method that updates an adaptive filter. The method includes receiving a reference signal representative of noise or vibration produced by a source and an error signal representative of the residual noise power in a target zone. The sub-band of the reference signal and the error signal are analyzed without identification of a secondary path. Finally, the adaptive filter coefficient is updated based on the sub-band analysis.

In another aspect, the present invention also relates to a method for reducing noise or vibration in a target zone. The noise or vibration is produced by a source and is transferred to the target zone by a main path. The method entails receiving a reference signal representative of a noise produced by a source and an error signal. The error signal represents the residual noise power in a target zone. The sub-bands of the reference signal and the error signal are then analyzed without identification of a secondary path. The adaptive filter coefficient is updated based on the analysis. Finally, a drive signal produced utilizing the adaptive filter coefficients is outputted to an actuator to provide an actuated signal into the target zone that reduces noise in the target zone.

In yet another aspect, the present invention relates to a control system for reducing noise or vibration in a target zone. The noise or vibration is produced by a source and is transferred to the target zone by a main path. The control system includes an actuator, at least one error sensor, and a controller. The actuator delivers actuated signals into at least a portion of the target zone. The error sensor monitors the residual noise or vibration power in the target zone and produces an error signal representative thereof. The controller receives a reference signal representative of noise or vibration produced by the source and the error signal. The error signal represents the residual noise power in the target zone. The controller analyzes of the reference signal and the error signal without identification of a secondary path. The controller also provides drive signals to the actuator to cause the actuator to deliver the actuated signals into the target zone so as to reduce a single-tone sinusoid or a multiple-frequency sinusoid in the target zone.

### BRIEF DESCRIPTION OF THE SEVERAL VIEWS OF THE DRAWING

So that the above recited features and advantages of the present invention can be understood in detail, a more particular description of the invention, briefly summarized above, may be had by reference to the embodiments thereof that are illustrated in the appended drawings. It is to be noted, however, that the appended drawings illustrate only typical embodiments of this invention and are therefore not to be considered limiting of its scope, for the invention may admit to other equally effective embodiments.

FIG. 1 is a block diagram of a feed-forward active control system using the Filtered-x LMS algorithm.

FIG. 2 is an expression of Equations (3) and (5) in the complex plane.

FIG. 3 is a geometric interpretation of Equation (11): move ∠S_{ω} out of ±90° range to inside of ±90° range.

FIG. 4 is a block diagram of algorithm steps in accordance with one version of the present invention.

FIG. 5a is a block diagram of ANC without secondary path identification for single-tone noise and narrowband noise, the dashed line representing a block that is only needed when the secondary path is time varying.

FIG. 5b is a block diagram of the hardware used to construct a control system in accordance with the present invention.

FIGS. 6a and 6b are exemplary Sub-band implementations of ANC without secondary path identification based on (a) Morgan's method [20] and (b) DeBrunner's method [22]. (The dashed line designates optional performance monitoring stage).

FIG. 7 is a flowchart of another example of broadband ANC without secondary path identification.

FIG. 8 is a flowchart of an example of broadband ANC without secondary path identification using adaptive sub-band selection.

FIG. 9 is a chart illustrating a phase response of the secondary path.

FIG. 10 are time sequence charts of the residual noise for the different algorithms. From top to bottom: the noise to be cancelled, the filtered-x LMS algorithm, the proposed algorithm in FIG. 5a, the basic LMS algorithm.

FIG. 11 Learning curve for our algorithm and full-band filtered-x LMS. (a) the filtered-x LMS algorithm, (b) the proposed control algorithm, (c) the frequency domain simultaneous perturbation method [11].

FIG. 12 is a chart illustrating an impulse response of the secondary path during 0˜210 s.

FIGS. 13(a) and (b) are charts illustrating phase response of the secondary paths, before (a) and after (b) change.

FIG. 14 is a learning curve of proposed algorithm for a sudden change of secondary path.

FIG. 15 is a learning curve of the proposed algorithm with changes in primary noise and additive noise powers.

FIG. 16 is a chart illustrating a phase response of the secondary path used for evaluating the adaptive sub-band selection technique.

FIG. 17 is a learning curve for adaptive sub-band selection and direction search stage. (a) Without ANC, estimate, ξ₁, eₘₐₓ and χ₁ (b) Update the adaptive filter using the low frequency component (the first sub-band) with positive step size, which reduces the excess noise power (c) Update the adaptive filter using the high frequency component (the second sub-band) with positive step size, which causes the adaptive filter to diverge (d) Update the adaptive filter using the high frequency component with negative step size, which still causes the adaptive filter to diverge (e) After splitting the high frequency sub-band into two sub-bands; update the adaptive filter using the (current) second sub-band component with a positive step size, and the adaptive filter converges (f) Update the adaptive filter using the (current) third sub-band component with a positive step size, (g) Update the adaptive filter using the (current) third sub-band component with a negative step size.

### DETAILED DESCRIPTION OF THE INVENTION

Presently preferred embodiments of the invention are shown in the above-identified figures and described in detail below. In describing the preferred embodiments, like or identical reference numerals are used to identify common or similar elements. The figures are not necessarily to scale and certain features and certain views of the figures may be shown exaggerated in scale or in schematic in the interest of clarity and conciseness.

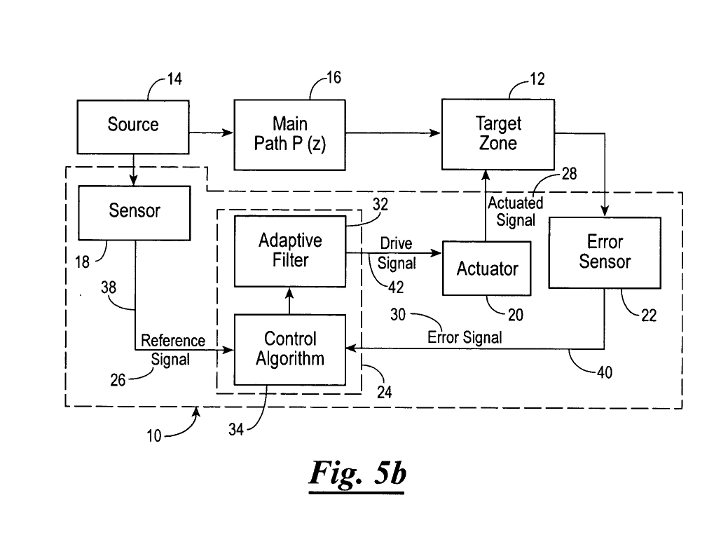

Referring now to the drawings, and in particular to FIGS. 5(a) and 5(b), shown therein are block diagrams of a control system 10 constructed in accordance with the present invention for reducing noise or vibration in a target zone 12. At least some of the noise of the target zone 12 is produced by a source 14 and transferred to the target zone 12 by a main path 16. The source 14 can be any device or apparatus that emits noise or vibration, such as a generator, an engine, industrial machinery, a road, ductwork, a plenum box, or a hydraulics system. The target zone 12 is any volume; area; or part of a device in which a noise can be felt or heard. Common examples of target zones 12 are a passenger area in a car, a container, a room, a muffler, or an inner part of a headphone. The main path 16 is a path that delivers the noise from the source 14 to the target zone 12. In general, the control system 10 has many uses, including the following: active noise cancellation headphones; noise reduction for machines (such as active silencers for large fan systems, washing machines, air conditioners); active exhaust mufflers; and in-vehicle noise reduction.

The control system 10 will be described hereinafter for noise reduction; however, the control system is equally applicable to vibration reduction. The noise control system 10 is includes one or more sensor 18, one or more actuator 20, one or more error sensor 22, and one or more controller 24. The sensor 18 detects the noise emitted from the source 14 and generates an analog or digital reference signal 26 representative of the noise. The sensor 18 can be any device or system for transforming noise into a reference signal 26. For example, the sensor 18 can be a microphone for detecting sound, or an accelerometer for detecting vibration. The actuator 20 delivers an actuated signal 28 into at least a portion of the target zone 12. The actuator 20 is any system or component that is capable of delivering the actuated signal 28 into the target zone 12 for reducing the noise or vibration from the source 14 transferred to the target zone 12 through the main path 16. For example, the actuator 20 can be a speaker for reducing noise, or one or more piezoelectric materials or solenoids for reducing vibration. The error sensor 22 monitors the residual noise power in the target zone 12. The error sensor 22 produces an error signal 30 representative of the residual noise power in the target zone 12. The error sensor 22 can be, but is not limited to, a sensor, device or any other system that can transform the residual noise power into a format usable by the controller 24. The controller 24 is programmed or hard coded to form an adaptive filter 32 controlled by a control algorithm 34. The control algorithm 34 of the controller 24 receives the reference signal 26 via a signal path 38, and the error signal 30 via a signal path 40. The control algorithm 34 of the controller 24 then preferably analyzes subbands of the reference signal 26 and the error signal 30 without identification of a secondary path (shown in FIG. 5a of embodiment 100), and provides adaptive filter coefficients to the adaptive filter 32 of the controller 24. The adaptive filter 32 of the controller 24 receives the reference signal 26, and the adaptive filter coefficients and provides drive signals 36 to the actuator 20 via a signal path 42 to cause the actuator 20 to deliver the actuated signals 30 into the target zone 12 so as to reduce the residual noise power in the target zone 12.

The controller 24 can be, but is not limited to, a microcontroller, a central processing unit, a digital signal processor and any associated hardware, such as D/A converters, A/D converters, amplifiers and the like. The controller 24 can be implemented as a single device, or multiple devices. The control algorithm 34 can be implemented as software or firmware stored on a computer readable medium, such as, a memory, hard drive, tape, optical medium, magnetic medium, and the like.

As discussed above, active noise control (ANC) has been widely applied in industry to reduce environmental noise and equipment vibrations. Most available control algorithms 36 require the identification of the secondary path, which increases the control system 10 complexity, contributes to an increased residual noise power, and can even cause the control system 10 to fail if the identified secondary path is not sufficiently close to the actual path. As discussed herein, based on the geometric analysis and the strict positive real (SPR) property of the filtered-x LMS algorithm, the controller 24 executes a new ANC control algorithm 34 suitable for single-tone noises as well as some specific narrowband noises that does not require the identification of the secondary path, though its convergence can be very slow in some special cases. We are able to extend the developed ANC control algorithm 34 to the case of active control of broadband noises through our use of a sub-band implementation of the ANC algorithm. Compared to other available control algorithms that do not require secondary path identification, the control algorithm 34 is simple to implement, yields good performance, and converges quickly. Simulation results confirm the effectiveness of the control algorithm 34.

### Example 1

### Single-Tone ANC without Secondary Path Identification

An example of an ANC control algorithm 34 without secondary path identification for a single-tone sinusoid noise is proposed in this section. In the real world, many noises are periodic, for instance, those that are generated by sources 14, such as engines, compressors, propellers, and fans [1]. As a result, the method in this Example does have some practical application. Also, as we shall see, this method can be directly extended to the parallel configuration for multiple-frequency ANC that was developed in [1, Sec. 4.4.2]. Meanwhile, the method from this Example is suitable for the active control of narrowband noise when the phase response of the secondary path meets a certain condition.

If the secondary path effect is not considered at all, the update of the adaptive filter coefficients w(n) based on the LMS algorithm is

w(n)=w(n−1)+μe(n)x*(n) (7)

where ε is a small positive number. In (7), the reference signal 26 does not need to pass through the secondary path. From the previous analysis, we find that for a signal-tone input X_{ω}(n)

W_{ω}(n)=W_{ω}(n−1)+μPₓ(ω)|S_{ω}|[P_{ω}/S_{ω}−W_{ω}(n−1)]e^{j∠S}^{_{ω}} (8)

where ∠S_{ω} represents the angle of S_{ω}, and |S_{ω}| represents the amplitude of S_{ω}. From the previous discussion and using (6), when the step size satisfies

$$μ < \frac{2 cos ( ∠ {S}_{ω} )}{{P}_{x} ( ω ) \vert {S}_{ω} \vert} \qquad ( 9 )$$

and the angle ∠S_{ω} is within the range of ±90°, the update of W_{ω}(n) is still appropriate, and convergence to the ideal value occurs even without secondary path identification. However, if ∠S_{ω} is outside of the range of ±90°, then the adaptive filter 32 W_{ω}(n) diverges, and the control system 10 will fail to cancel the single-tone noise [19]. In this case, if the updating equation is changed from (7) by changing the sign in front of μ from a minus to a plus, i.e., if

w(n)=w(n−1)−μe(n)x*(n) (10)

is used then for a single-tone input,

W_{ω}(n)≈W_{ω}(n−1)+μPₓ(ω)|S_{ω}|[P_{ω}/S_{ω}−W_{ω}(n−1)]e^{j}(∠S^{_{ω}}^{-108°)}* (11)

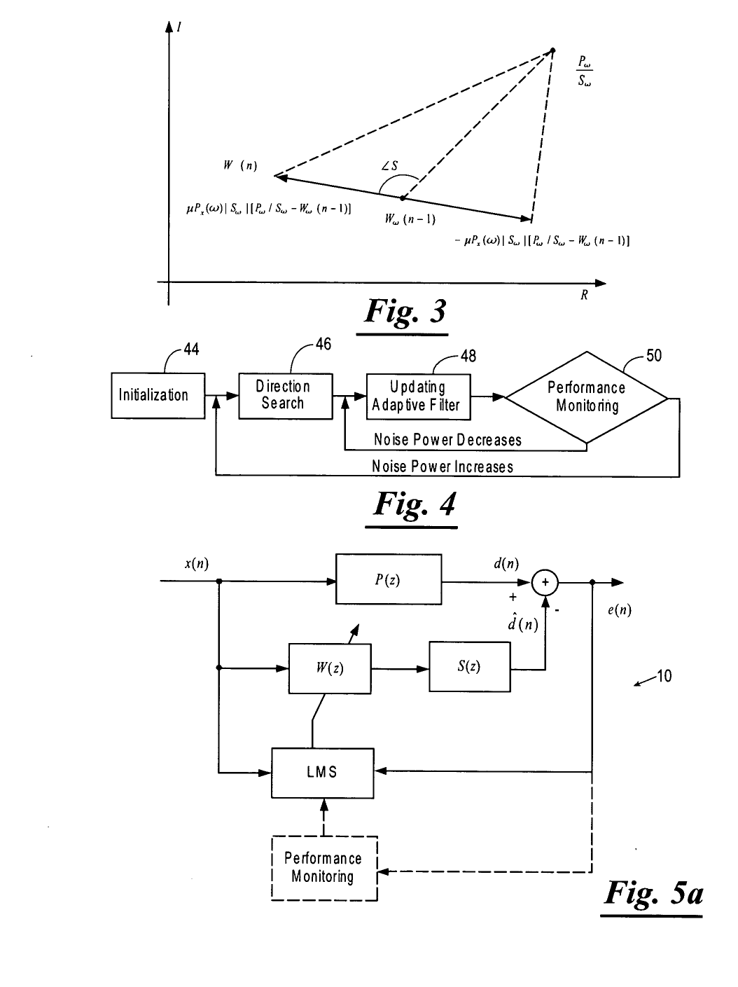

By changing the direction of the step μ (equivalently, by changing the sign in front of μ in the update equation), the angle difference is moved from outside the ±90° range to inside the ±90° range, which ensures that the SPR property is met. This consequence is illustrated in FIG. 3. By exploring this property and assuming that the phase response ∠S_{ω} of the secondary path is known, Bjarnason et al [9] introduced an adaptive control algorithm for narrowband noise that does not require full identification of the secondary path. Note that the estimation of the phase response of the secondary path is still required. However, that method does not work when 1) the correct phase response of the secondary path is unavailable or 2) the secondary path is time varying.

Without up-to-date information on the secondary path, the controller 24 cannot know if ∠S_{ω} is inside the allowable phase range of ±90°, or whether it is outside this range. Consequently, the controller 24 does not know when to change the sign in front of μ to yield a converging adaptive filter 32. In this paper, we propose a method to determine the appropriate sign as the adaptive filter 32 runs. The following assumption is used:

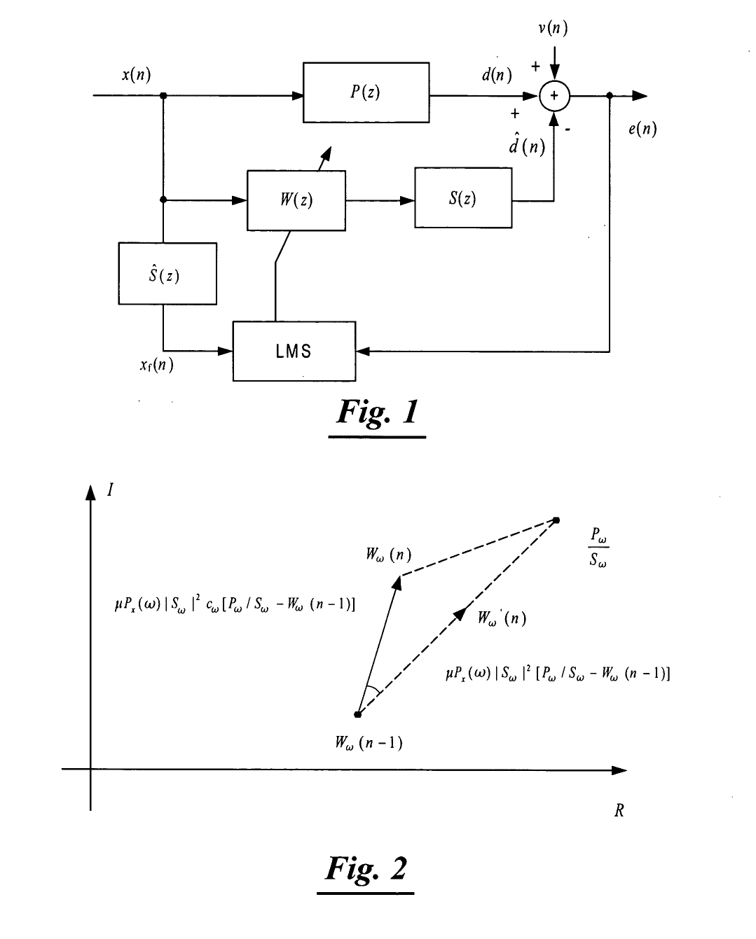

Assumption 1. The additive noise v(n) in FIG. 1 is wide-sense stationary or varying slowly with known power range Pₘₐₓ/Pₘᵢₙ=c, where Pₘₐₓ and Pₘᵢₙ represent, respectively, the maximum and the minimum instantaneous power of v(n).

The additive noise powers Pₘₐₓ and Pₘᵢₙ may be determined experimentally by turning off the input reference and then directly measuring the additive noise. Using this practical assumption, we propose a new algorithm for the active control of single-tone noise that does not require any identification of the secondary path as follows (shown in FIG. 4):

Initialization stage 44:
1. Initialize the adaptive filter coefficient vector w(n) with zeros, the number of samples data, N, used for estimating the noise power, the step size μ, the fluctuation factors δ₁ and δ₂, and the variation factor c′=max {c,1+δ₁}. The small positive constants δ₁ and δ₂ provide algorithmic tolerance to the power estimates.
   Direction search stage 46:
2. Without updating the adaptive filter coefficients, measure the mean noise power

$${ξ}_{1} = {\sum }_{i = 0}^{N - 1} {e}^{2} ( i ) ,$$

maximum noise amplitude eₘₐₓ=max (|e(i)|), and reference noise power

$${X}_{1} = {\sum }_{i = 0}^{N - 1} {x}^{2} ( i )$$

for the N samples.
3. Update the adaptive filter 32 using (7) and measure the mean noise power ξ₂ and mean reference noise power χ₂ as in Step 2 for another N samples, or stop the updating if |e(i)|>(1+ξ₂)eₘₐₓ.
4. If ξ₂/χ₂>ξ₁/χ₁ or |e(i)|>(1+δ₂)eₘₐₓ, change the sign of μ.
   Updating stage 48:
5. Update the adaptive filter 32 using (7).
   Performance monitoring 50 stage (for a system with a time-varying secondary path):
6. Initialize n=1, χ(0)=χ₁ and ξ(0)=ξ₁.
7. Calculate the mean noise power (n) and mean reference signal 26 power χ(n) iteratively using ξ(n)=λξ(n−1)+e²(n) and χ(n)=λχ(n−1)+x²(n), where λ is a forgetting factor in the range λε[0.5,1). Usually,

$$1 - \frac{1}{2 L} < λ < 1 ,$$

where L is the effective data length used in estimation.

If ξ(n)/χ(n)>(1+ξ₁)c′ξ(n−N)/χ(n−N) or ξ(n)/χ(n)>c′ξ₁/χ₁, then go to Step 2 and redo the direction search; otherwise, go to step 5 and keep updating.

This algorithm can be divided into four stages, i.e., initialization 44, direction search 46, updating 48, and performance monitoring 50, as shown in FIG. 4. As we have seen, the significant issue for the algorithm is the choice of the right sign of μ—the proper convergence direction of the adaptive filter coefficients. This issue is addressed by first initializing 44 the step size with a sufficiently small positive value μ. Then, the controller 24 monitors the excess noise power. If the noise power increases, then it is assumed that the adaptive filter coefficients are moving to increase the error, and so the sign in front of μ in the update equation is changed 46. After determining the correct direction 46, the control algorithm 34 has a structure similar to the filtered-x LMS algorithm, but the reference signal 26 does not need to be processed by the estimated secondary path (see the block diagram for our algorithm in FIG. 5a).

At initialization 44, the adaptive filter coefficient vector w(n) is set to zero, for example. The number of samples of data, N, used to estimate the noise power is set according to the frequency of the reference signal 26 as well as the variance of the additive noise v(n). The variation factor c′ is given by

c′=max{c,1+δ₁} (12)

where c is defined in Assumption 1, and the small positive number δ₁ inoculates the algorithm against errors in estimating the residual noise power. A second fluctuation factor δ₂ also provides similar tolerance to estimation errors for the maximum residual noise amplitude. The choice of these two fluctuation factors depend on N and the distribution of the additive noise. With a good choice for these fluctuation factors the control algorithm 34 will tolerate estimate errors while remaining sensitive to any changes in the secondary path.

When the secondary path is stationary, the adaptive filter 32 can be updated after determining the right update direction 48 without using the performance monitoring 50 stage. Doing so will reduce the system complexity. Also, we can eliminate measuring the reference signal 26 mean power when the reference noise is wide-sense stationary, because χ₁ and χ(n) in the direction search and performance monitoring 50 stages are then constant.

Using the geometric analysis technique, this method can be applied to narrowband noise—or even broadband noise—if at a particular frequency band, the secondary path phase response is such that

−90°+k×180°<∠S_{ω}<90°+k×180° (13)

where k is an arbitrary integer, and ω is in the noise bandwidth. The condition (13) is equivalent to the ±90° stability bound and the SPR property of the filtered-x LMS algorithm. However, according to the discussion in [23, Sect. 2.6.3], this SPR condition can be relaxed. We find that the adaptive filter 32 will asymptotically converge even when the SPR condition of Eq. (13) is satisfied only at the frequency range where the noise to be cancelled has dominant energy. Because the majority of frequency components of the reference noise satisfy (13), the adaptive filter 32 W_{ω}(n), will, for most frequencies, move closer to the expected value P_{ω}/S_{ω}compared to W_{ω}(n−1) as shown in FIGS. 2 and 3. The adaptive filter 32 W_{ω}(n) will diverge from its expected value P_{ω}/S_{ω}for only a very few frequency components. As long as the range of frequencies for with the filter converges is more significant than the range of frequencies where the filter does not converge, then the adaptive filter 32 will converge in a statistical sense. Consequently, if the phase response of the secondary path almost satisfies (13), then the adaptive filter 32 updated by either (7) or (10) will converge. Simulation results are provided below that indicate the validity of this heuristic argument.

The upper bound for the step size for our proposed ANC algorithm with a narrow-band or broad-band noise that meets (13) can be obtained from (9) as

$$μ < {min}_{ω} \frac{2 \vert cos ( ∠ {S}_{ω} ) \vert}{{P}_{x} ( ω ) \vert {S}_{ω} \vert} . \qquad ( 14 )$$

However, without any secondary path (shown in FIG. 5a) information, we can only approximate the largest appropriate step size by experimentation. Otherwise, a relatively small step size is used, which of course reduces the convergence speed of the adaptive filter 32. However, judicious use of some prior information about the secondary path can help choose larger, but still appropriate, step sizes. For example, one could use the approximate secondary path magnitude (or phase) response range within a sub-band to determine approximate step sizes. Note that step size should always be that of the sub-band with the least upper bound to ensure convergence. In practice, some of this information is available when an ANC or AVC system is set up.

In one extreme situation for single-tone noise, if ∠S_{ω} happens to equal ±90°+k×180°, then no matter what sign the step size takes, our adaptive filter 32 will never converge. One way to solve this problem is through adding delay to the reference signal 26 that pushes the phase outside of the ±90° area. In most cases, this problem is unimportant because not every frequency component will be exactly ±90°, and so the other components will drive the convergence of the filter, as discussed in the text following (13).

### Example 2

### Broadband ANC without Secondary Path Identification

Though the algorithm of Example 1 for single-tone noise without secondary path identification has a few practical applications, when the noise to be cancelled is broadband, or narrowband but the secondary path phase response doesn't meet the requirement of (13), then that method is not appropriate. In this section, a new ANC method is introduced for these situations that also does not require the identification of the secondary path. This method desirably uses a sub-band implementation of the ANC techniques, i.e., converting the broadband ANC problem into several narrowband noise control problems that are suitable for treatment by the method developed in Example 1.

### A. Sub-Band Implementation of ANC

Delayless sub-band ANC algorithms are discussed in [20]-[21] to overcome the slow convergence of the filtered-x LMS algorithm caused by the wide spectral dynamic range of the reference signal 26. The method introduced by Morgan et al [20] can even reduce the computational complexity by approximately the number of sub-bands used for high-order adaptive filters 34. Park et al [21] further improved Morgan's method by decomposing the secondary path into a set of sub-band functions. The newly introduced sub-band ANC algorithm by DeBrunner et al [22] does not require the up-sampling and down-sampling in the sub-bands as in [20], [21], which is more efficient for lower-order adaptive filters 34, and does not require perfect sub-band filters, because reconstruction is not performed.

B. Sub-Band Implementation of ANC without Secondary Path Identification

By employing either of the methods introduced in [20] or [22], we can divide the broadband signal into narrowband signals. Choosing enough sub-bands makes each sub-band signal meet the condition in (13). Then we apply the method discussed in Example 1 to each sub-band.

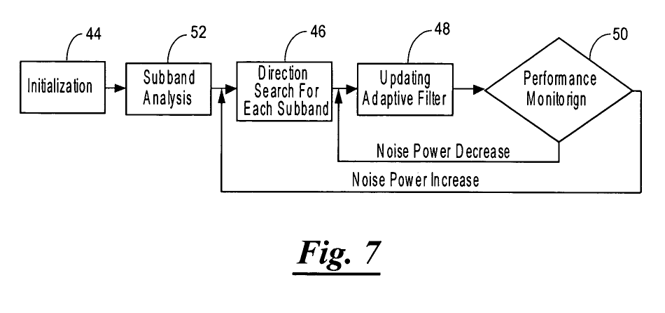

The sub-band implementation of ANC without secondary path (shown in FIG. 5a) identification is shown in the block diagram of FIG. 7, and detailed as follows:
1. Sub-band analysis of reference and error signals 32 as in either [20] or [22] (as indicated in FIG. 7 by the reference numeral 52).
2. Determine the appropriate update direction in each sub-band. To avoid sub-band interference, the controller 24 finds one sub-band direction at a time. Consequently, in the direction search stage, the controller 24 only updates the coefficients for one sub-band in Morgan's sub-band configuration [20], or updates the adaptive filter coefficients based on one sub-band reference signal 26 and error signal 30 in DeBrunner's configuration [22].
3. Update 48 the adaptive filter 32 while monitoring 50 the system performance. This is done precisely as described in Example 1. When the performance deteriorates, the controller 24 redos Step 2 using the alternative direction.

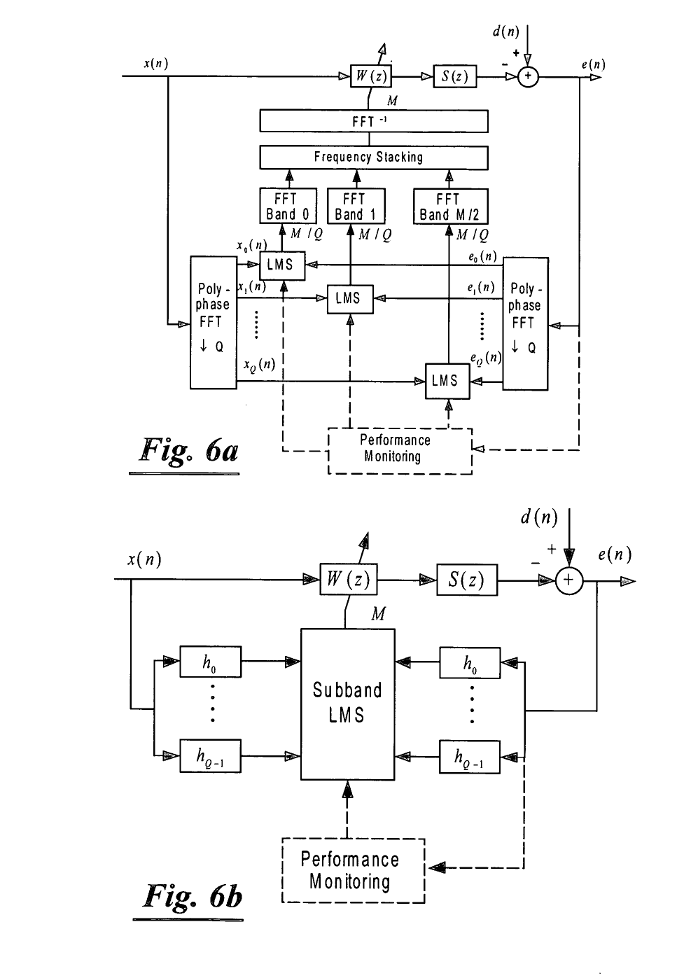

A block diagram of the proposed algorithm based on Morgan's sub-band configuration is shown in FIG. 6 (a) while that based on DeBrunner's configuration is shown in FIG. 6 (b). A flowchart of the proposed algorithm is given in FIG. 7. The number of sub-bands can be a critical factor. If the approximate phase response of the secondary path is known, the controller 24 can choose a filter bank that guarantees that the phase response of each sub-band secondary path meets or almost meets the constraint of (13). In cases where the phase is completely unknown, the controller 24 uses many sub-bands; sometimes, maybe more than necessary.

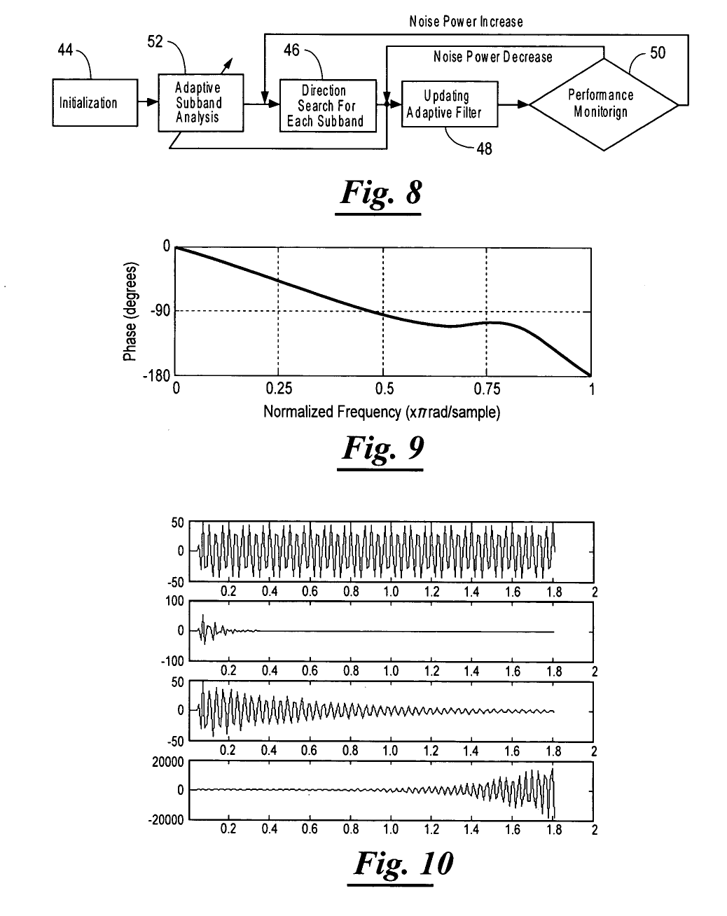

### C. Adaptive Sub-Band Selection 54 (Shown in FIG. 8)

Without any information about the secondary path, the controller 24 chooses more sub-bands than are really required, thus ensuring that the algorithm works. Increasing the number of sub-bands in the Morgan configuration leads to higher decimation rates with a corresponding larger lag in convergence. In the DeBrunner configuration, increasing the number of sub-bands increases the computational complexity.

Also, since the control algorithm 34 determines the adaptive filter 32 direction for each sub-band, increasing the number of sub-bands in the control algorithm 34 corresponds to increasing the time spent in determining the appropriate search directions. Consequently, the control algorithm 34 is desirably provided with an adaptive sub-band selection method that seeks to minimize the required number of sub-band signals that must be used. At the sub-band analysis stage, the control algorithm 34 guesses at the number of sub-bands required to do the analysis. Then, the control algorithm 34 determines the appropriate direction for each sub-band by updating each sub-band in turn with a positive, but sufficiently small step size p, for which the adaptive filter 32 converges if condition (13) is met. If the residual noise power increases, then the control algorithm 34 redos the update for that particular sub-band by toggling the sign of p. If the residual noise power still increases, then the control algorithm 34 assumes that the phase response of the secondary path in this sub-band doesn't satisfy (13). In this case, the control algorithm 34 increases the number of sub-bands (by splitting the current one) and determines the appropriate direction for each newly created sub-band. A flowchart of the proposed algorithm 34 combined with adaptive sub-band selection is shown in FIG. 8. This method will introduce unevenly distributed sub-band filters.

### Computational Complexity Analysis

In this section, computational complexity analyses for the derived control algorithms 34 are provided. The comparison uses the number of real multiplications per iteration during the update stage for the different algorithms. For the direction search and the adaptive sub-band selection stages, the computational complexity for one iteration can be approximated by the computational complexity during the update stage divided by the number of sub-bands since the control algorithm 34 typically only updates one sub-band at a time. In the following calculation, M is the length of the adaptive control filter, K is the length of the secondary path FIR filter model, Q represents the number of sub-bands in the DeBrunner configuration (which is equivalent to a 2Q-point FFT in the Morgan algorithm), L is the length of the sub-band filters, and P is the number of taps for the prototype convolution filter in Morgan's algorithm.

For a single-tone or narrowband ANC system that satisfies the constraint given in (13), the conventional filtered-x LMS algorithm requires 2M+3K+1 multiplications (the on-line identification of the secondary path requires 2K multiplications). The proposed control algorithm 34 requires 2M+7 multiplications (the performance monitoring 50 requires 6 multiplications). Significant computational savings in the proposed control algorithm 34 are found for this case.

For broadband ANC, the proposed control algorithm 34 could have at least two configurations: one based on the Morgan configuration shown in FIG. 6 (a), and another based on the DeBrunner configuration shown in FIG. 6 (b). The number of multiplications for the different algorithms is given in Table 1. For example, assuming M=512, K=256, P=128, and Q=16 as given in [20]: the number of real multiplications required for the filtered-x LMS algorithm is 1793 per iteration; for the DeBrunner Algorithm is 17680 per iteration; for the proposed control algorithm 34 based on the DeBrunner configuration is 16918 per iteration; for the Park algorithm is about 1110 per iteration; and for the proposed control algorithm 34 based on the Morgan configuration is 701 per iteration. Remember that in any practical implementation, the engineer must weigh computational complexity with performance. The DeBrunner configuration usually yields the fastest convergence without lag in convergence, while the Morgan configuration can provide good performance with low computational complexity. No matter which sub-band configuration is use, significant computational savings using the proposed control algorithms 34 are achieved, due to removal of the secondary path estimation and the associated filtering of the reference signal 26 with the estimated secondary path.

TABLE 1
MULTIPLICATION COMPARISONS OF DIFFERENT ALGORITHMS.

| Algorithm | Number of multiplications for one iteration |
|---|---|
| The filtered-x LMS algorithm with on-line identification | 2M + 3K + 1 |
| Sub-band filtered-x LMS with on-line identification (DeBrunner Algorithm [22]) | Q(2K + M + 1) + M + 3K |
| Sub-band filtered-x LMS with on-line identification (Park Algorithm [20, FIG. 1], which is derived from the Morgan Configuration) | $\begin{matrix}M + \frac{2 ( P + 2 M + 12 K )}{Q} + \frac{4 ( M + 6 K )}{{Q}^{2}} + \\ 2 {log}_{2} ( 2 Q ) + 3 {log}_{2} ( M ) + \frac{2}{Q} {log}_{2} \frac{K}{Q}\end{matrix}$ |
| The proposed control algorithm 34 with performance monitoring 50 based on the DeBrunner configuration | Q(2K + M + 1) + M + 6 |
| The proposed control algorithm 34 with performance monitoring 50 based on the Morgan configuration | $M + \frac{2 ( P + 2 M )}{Q} + \frac{4 M}{{Q}^{2}} + 2 {log}_{2} ( 2 Q ) + 3 {log}_{2} ( M )$ |

Simulation Results

Here several simulation results are provided to show the effectiveness of the proposed control algorithms 34. Different proposed control algorithms 34 performance are compared in term of residual noise power:

Residual Noise Power (dB)=10 log₁₀E[e²(n)]

or normalized residual noise power (NRNP):

$$NRNP ( d B ) = 10 {log}_{10} \frac{E [ {e}^{2} ( n ) ]}{E [ {d}^{2} ( n ) ]} .$$

### Simulation 1. Stationary Secondary Path for Single-Tone ANC

In this simulation, an ANC system is sampled at a rate of 100 Hz, the main path 16 is modeled by an FIR filter with impulse response

h(n)=δ(n−3)−2.7083δ(n−4)+4.1861δ(n−5)−3.0451δ(n−6)+0.73071δ(n−7)

and the secondary path is modeled by an IIR filter with transfer function

$$\frac{{z}^{- 1} + 0.96 {z}^{- 2} + 0.4923 {z}^{- 3}}{1 + 1.06 {z}^{- 1} + 0.3352 {z}^{- 2}}$$

The phase response of this secondary path is shown in FIG. 9. Note that, throughout this and the following simulations, we assume the secondary path information is unavailable. The reference signal 26 is a sine wave whose frequency is 30 Hz. The adaptive filters 34 (with order 1), based on the filtered-x LMS algorithm, the proposed control algorithm 34 with configuration as in FIG. 4, and the LMS algorithm—without considering the secondary path effect, as in (7)—are implemented, respectively. The step sizes are set to the largest value possible while still assuring that the adaptive filters 34 converge. The residual noises after the adaptive control filter converges for the different algorithms are shown in FIG. 10.

From this simulation, as expected, for single-tone noise, the filtered-x LMS converges much faster than does one version of the proposed control algorithm 34 and that the ANC based on LMS algorithm will diverge. We also notice that if the reference noise possesses frequency content around 22 Hz, the version of the proposed control algorithm 34 will converge very slowly or will not converge, because the phase response of the secondary path is close to −90°. However, as we discussed in Example 1, by adding a unit sample delay in the reference signal 26, the slow convergence will be significantly improved.

### Simulation 2. Broadband ANC for Stationary Secondary Path

In the simulation, the ANC system has the same configuration as in Simulation 1, except that reference noise and additive noise are white, Gaussian, and stationary; and the adaptive filter 32 order increases to 48. We implement the full-band normalized filtered-x LMS algorithm, the frequency domain simultaneous perturbation algorithm with L=100, a=0.5 (the same notation as in [11]), and the proposed control algorithm 34 as in FIG. 6 (b) using four the linear phase paraunitary filter bank described in [24, Table II]. Note that, from the phase response shown in FIG. 9, two sub-bands will be sufficient for the convergence of the proposed control algorithm 34; however, without any information about the secondary path, we tend to use more sub-bands than necessary, as discussed in Example 2. The measurement signal to noise ratio (SNR) is 20 dB. In our simulations, the fluctuation factors δ₁ and δ₂ are both 0.2, so we have c′=1.2 from (12); the forgetting factor A is 0.995. FIG. 11 shows the learning curves for the different algorithms at their fastest convergence speed, based on an ensemble average of 200 runs.

We find that all algorithms can effectively reduce the noise. However, without the secondary path information, the proposed control algorithm 34 converges at a slower speed than does the filtered-x LMS, but still much faster than the frequency domain simultaneous perturbation method [11], which converges after 60,000 iterations, with a slightly higher residual noise power due to the perturbation.

### Simulation 3. Sudden Change in the Secondary Path

We simulate an ANC system where the main path 16 is modeled by an FIR filter with impulse response:

h(n)=2δ(n−3)−1.7083δ(n−4)+3.1861δ(n−5)−2.0451δ(n−6)+1.73071δ(n−8)

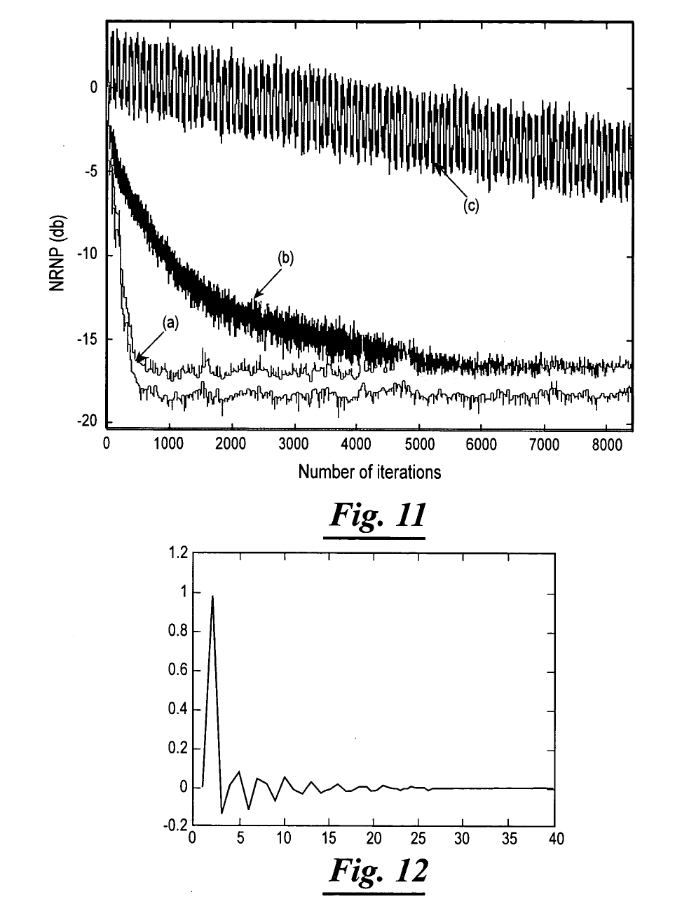

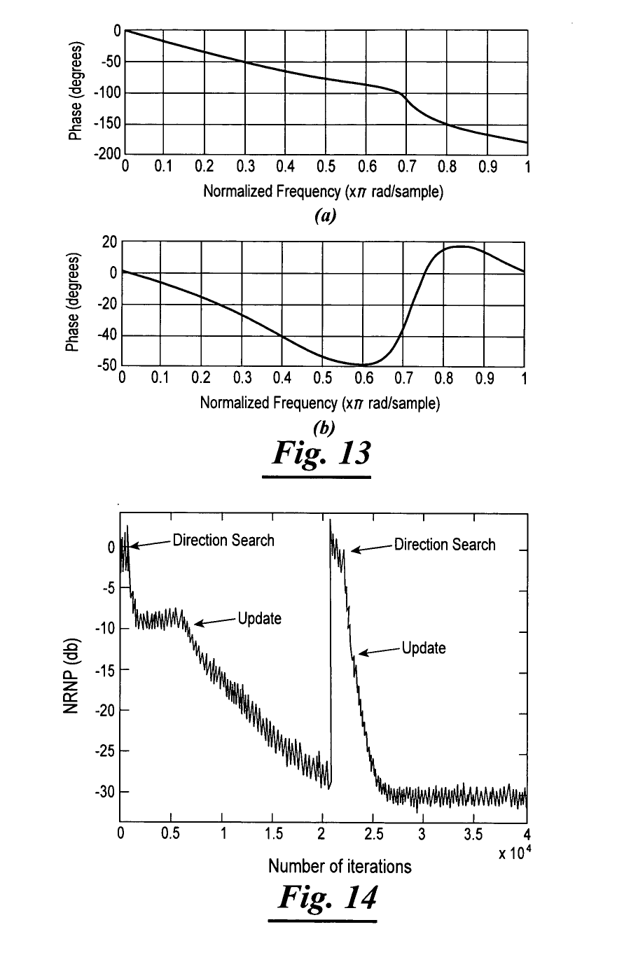

and the secondary path has an impulse response shown in FIG. 12 till 210 s, whose phase response is shown in FIG. 13(a). After that time, the secondary path changes to an FIR filter with impulse response:

h(n)=δ(n)+0.7δ(n−1)+0.3352δ(n−2)−0.2δ(n−3)+0.02δ(n−4)

whose phase response is shown in FIG. 13 (b). Again, the proposed control algorithm 34 according to FIG. 6 (b) is implemented with the same four sub-bands as in Simulation 2. The measurement noise is set to 32 dB and the remaining simulation parameters are unchanged from those used in Simulation 2. The direction search for each sub-band takes 2 s, i.e., N=200 samples. The learning curve for an average of 200 runs is shown in FIG. 14. From this figure, we find that our algorithm is robust with respect to a sudden change in the secondary path. The filtered-x LMS algorithm needs an online secondary identification configuration to handle this situation. However, as the simulations in [7] show, most ANC systems with on-line secondary path identification will diverge without any other constraints.

### Simulation 4. Changes in the Primary Noise and Additive Noise Power

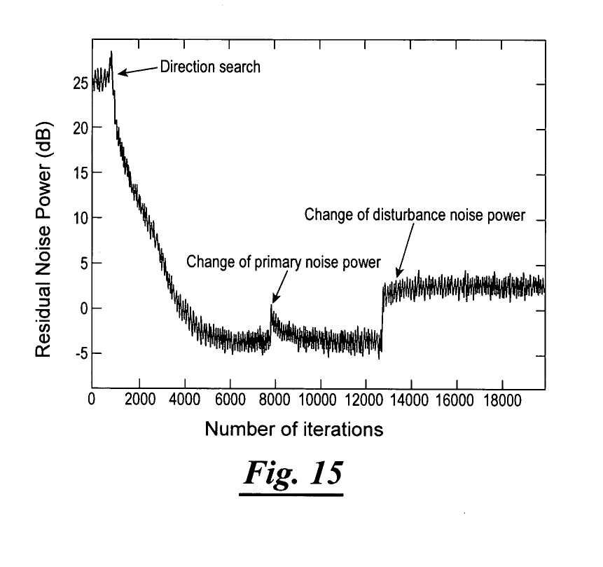

The ANC system has the same parameters as in Simulation 2, except that at time 80 s (after the adaptive filter 32 converges) and 130 s, there are 6 dB increases in the primary noise power and the additive noise power, respectively. As a result, we choose c′=4 from (12). The forgetting factor λ we use is 0.995, N=200, and the fluctuation factors δ₁ and δ₂ both remain at 0.2. The learning curve for an average of 200 runs is shown in FIG. 15. From this figure, we see that the proposed control algorithm 34 tolerates the changes in both the primary noise power and the additive noise power. We also notice that if there is an error in the estimation of Pₘₐₓ/Pₘᵢₙ=c (say if the estimated c=3), then at time 130 s the proposed control algorithm 34 will generate a false direction search request. On the other hand, if the estimated c is greater than the actual c, the ANC system will require more time to respond to any sudden change in the secondary path. These simulation results are not shown here.

### Simulation 5. Adaptive Sub-Band Selection Technique

In order to demonstrate the adaptive sub-band selection technique, the secondary path is modeled by an FIR filter with impulse response

h(n)=δ(n)+0.8δ(n−1)−1.2δ(n−2)

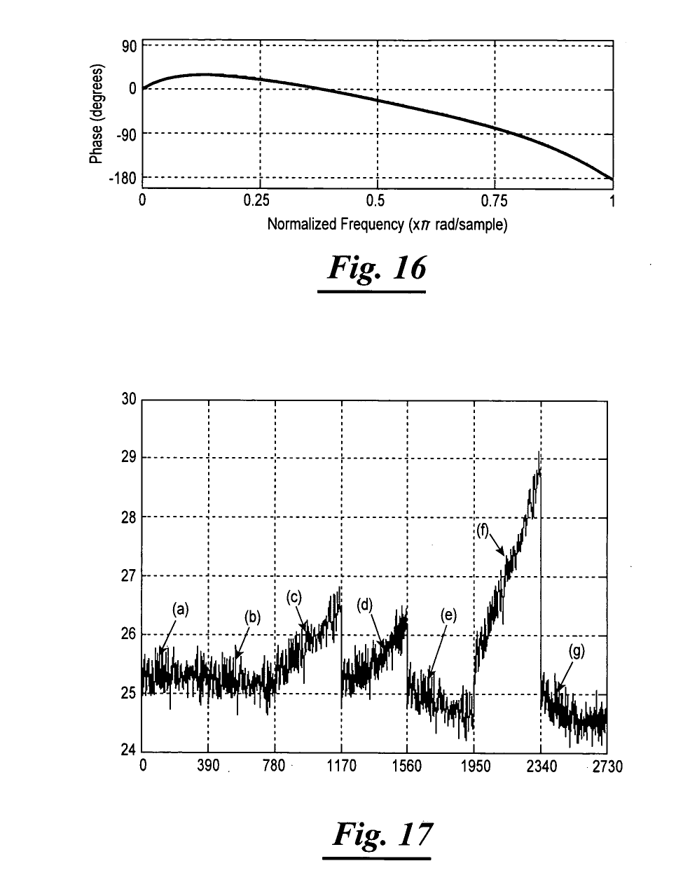

whose phase response is shown in FIG. 16. We start with two sub-band filters, one a lowpass FIR filter with coefficient [0.1629, 0.5055, 0.4461, −0.0198, −0.1323, 0.0218, 0.0233, −0.0075]; and the other a highpass filter with cutoff frequencies at half of the Nyquist frequency and coefficient [0.1629, −0.5055, 0.4461, 0.0198, −0.1323, −0.0218, 0.0233, 0.0075]. Assuming no knowledge of the secondary path, the step sizes for each sub-band are the relatively small value 0.02. FIG. 17 shows the average learning curve of 500 runs for the adaptive sub-band selection and direction search stages for each sub-band. In this simulation, N is 390, δ₁ is 0.2, and δ₂ is set at 10. Note that, in order to align the learning curve for each run, we have set δ₂ to a large value. This ensures that each sub-band direction search takes N iterations. From this simulation, two sub-band configurations will not work in this case because the adaptive filter 32 cannot reduce the high-frequency noise components using either ±μ. Therefore, we split the high frequency component into two more sub-bands. Here, we use the same sub-band filters as in [24, Table II]. This time, the control algorithm 34 successfully finds a correct direction for each sub-band, thus reducing the residual noise power. Thus, convergence requires the unevenly distributed sub-bands, where the low-pass sub-band has a cut-off at 0.5 normalized frequency, the high-pass sub-band now has a cut-off frequency at 0.75 normalized frequency, and thus, we have a band-pass sub-band lying between these two sub-bands. While the two lower frequency sub-bands satisfy (13), note that the third sub-band does not. However, even so, the adaptive filter 32 still converges.

From these simulation results, we observe that our algorithm converges more slowly than does the filtered-x LMS algorithm. However, this faster convergence of the filtered-x LMS is based on the correct estimation of the secondary path, and is not robust to errors in the estimation of that secondary path. In cases where there are errors in its estimate, or where it unexpectedly changes, the convergence speed of the filtered-x LMS will also be slow, or the algorithm could even diverge. The relatively slower convergence of the proposed control algorithm 34 is justified by the low residual noise and its robustness.

Thus, the filtered-x LMS algorithm was analyzed and the ±90° bound (SPR) property was pointed out from a geometric point of view. With this new insight, we first proposed a new ANC control algorithm 34 without secondary path identification for the active control of a single-tone noise and certain narrowband noises (see Example 1), though it may convergence very slowly in some special cases. Then, the control algorithm 34 was extended to control broadband noise by employing a sub-band implementation of the ANC algorithm (see Example 2). The control algorithms 34 outperform the available related algorithms in either convergence rate, implementation cost, or both. Compared to the conventional filtered-x LMS, the proposed control algorithms 34 require considerably fewer computations and offer greater configuration simplicity. However, as we found using FIGS. 2 and 3 and observed in our simulation results, without secondary path identification our proposed adaptive filter 32 does not converge toward the optimum value in the quickest manner. Consequently, the versions of the proposed control algorithms 36 simulated reduce the convergence speed when compared to the filtered-x LMS algorithm with full secondary path identification.

### REFERENCES

The portions of the following references referred to above are hereby incorporated herein by reference.
[1] S. M. Kuo and D. R. Morgan, Active Noise Control Systems: Algorithm and DSP Implementations. New York: Wiley, 1996.
[2] S. Elliott, Signal Processing for Active Control. San Diego: Academic Press, 2001.
[3] C. Bao, P. Sas, and H. V. Brussel, “Adaptive active control of noise in 3-D reverberant enclosures,” J Sound Vib., vol. 161, pp. 501-514, March 1993.
[4] S. M. Kuo and D. Vijayan, “A secondary path modeling technique for active noise control systems,” IEEE Trans. Speech Audio Processing, vol. 5, pp. 374-377, July 1997.
[5]H. Lan, M. Zhang, and W. Ser, “An active noise control system using online secondary path modeling with reduced auxiliary noise,” IEEE Signal Processing Lett., vol. 9, pp. 16-18, January 2002.
[6] M. Zhang, H. Lan, and W. Ser, “Cross-updated active noise control system with online secondary path modeling,” IEEE Trans. Speech Audio Processing, vol. 9, pp. 598-602, July 2001.
[7] M. Zhang, H. Lan, and W. Ser, “A robust online secondary path modeling method with auxiliary noise power scheduling strategy and norm constraint manipulation,” IEEE Trans. Speech Audio Processing, vol. 11, pp. 45-53, January 2003.
[8] P. L. Feintuch, N. J. Bershad, and A. K. Lo, “Frequency domain model for ‘filtered’ LMS algorithms. Stability analysis, design, and elimination of the training mode,” IEEE Trans. Signal Processing, vol. 41, pp. 1518-1531, April 1993.
[9] E. Bjarnason. “Algorithms for active noise cancellation without exact knowledge of the error-path filter,” in Proc. of the 1994 IEEE International Symposium on Circuits and Systems. 1994, pp. 573-576.
[10] Y. Kajikawa and Y. Nomura, “Active noise control system without secondary path model,” Electron. Commun. Jap., Part III, vol. 83, pp. 47-55, April 2000.
[11] Y. Kajikawa and Y. Nomura, “Active noise control without a secondary path model by using a frequency-domain simultaneous perturbation method with variable perturbation,” in Proc. 2003 IEEE International Conference on Acoustics, Speech, and Signal Processing, 2003, vol. V, pp. 580-583
[12] K. Fujii, M. Muneyasu, and J. Ohga, “Active noise control system using the simultaneous equation method without the estimation of error path filter coefficients,” Electron. Commun. Jap., Part III, vol. 85, pp. 101-108, June 2002.
[13] A. Sano and Y. Ohta. “Adaptive active noise control without secondary path identification,” in Proc. 2003 IEEE International Conference on Acoustics, Speech, and Signal Processing, 2003, vol. VI, pp. 213-216.
[14] Y. Ohta, T, Kohno, H. Ohmori, and A. Sano, “Direct fully adaptive active noise control algorithms without identification of secondary path dynamics,” in Proc. of the 2002 IEEE International Conference on Control Applications, 2002, pp 453-458.
[15] D. Zhou, and V. DeBrunner, “ANC algorithms that do not require identifying the secondary path,” in Proc. 2005 IEEE International Conference on Acoustics, Speech and Signal Processing, 2005, vol. III pp. 125-128.
[16] S. D. Snyder and C. H. Hansen, “Effect of transfer function estimation errors on the filtered-X LMS algorithm,” IEEE Trans. Signal Processing, vol. 42, pp. 950-953, April 1994.
[17] W. Ren and P. R. Kumar, “Adaptive Active Noise Control: Structure, Algorithms and Convergence Analysis,” in Proc. Inter-Noise, 1989, pp. 435-440.
[18] N. Saito and T. Sone, “Influence of modeling error on noise reduction performance of active noise control systems using filtered-X LMS algorithm,” J. Acoust. Soc. Jap. (E), vol. 17, pp. 195-202, July 1996.
[19] D. R. Morgan, “An analysis of multiple correlation cancellation loops with a filter in the auxiliary path,” IEEE Trans. Acoust., Speech, Signal Processing, vol. ASSP-28, pp. 454-467, August 1980.
[20] D. R. Morgan and J. C. Thi, “A delayless sub-band adaptive filter architecture,” IEEE Trans. Signal Processing, vol. 43, pp. 1819-1830, August 1995.
[21] S. J. Park, J. H. Yun, Y. C. Park, and D. H. Youn, “A delayless sub-band active noise control system for wideband noise control,” IEEE Trans. Speech Audio Processing, vol. 9, pp. 892-899, November 2001.
[22] V. DeBrunner, L. DeBrunner, and L. Wang, “Sub-band adaptive filtering with delay compensation for active control,” IEEE Trans. Signal Processing, vol. 52, no. 10, pp. 2932-2941, October 2004.
[23] B. D. O. Anderson, R. R. Bitmead, C. R. Johnson Jr., P. V. Kokotovic, R. L. Kosut, I. M. Y. Mareels, L. Praly, and B. D. Riedle, Stability of Adaptive Systems: Passivity and Averaging Analysis. Cambridge, Mass.: MIT Press, 1986.
[24] A. K. Soman, P. P. Vaidyanathan, T. Q. Nguyen, “Linear phase paraunitary filter banks: theory, factorizations and designs,” IEEE Trans. on Signal Processing, vol. 41, no. 12, pp. 3480-3496, December 1993.

It will be understood from the foregoing description that various modifications and changes may be made in the preferred and alternative embodiments of the present invention without departing from its true spirit. The devices included herein may be manually and/or automatically activated to perform the desired operation. The activation may be performed as desired and/or based on data generated, conditions detected and/or analysis of results.

This description is intended for purposes of illustration only and should not be construed in a limiting sense. The scope of this invention should be determined only by the language of the claims that follow. The term “comprising” within the claims is intended to mean “including at least” such that the recited listing of elements in a claim are an open group. “A,” “an” and other singular terms are intended to include the plural forms thereof unless specifically excluded.

1. A control system for reducing noise or vibration in a target zone, the noise or vibration produced by a source and transferred to the target zone by a main path, the control system, comprising:
an actuator positioned to deliver actuated signals into at least a portion of the target zone;
at least one error sensor monitoring the residual noise or vibration power in the target zone and producing an error signal representative thereof; and
a controller receiving a reference signal representative of noise or vibration produced by the source, and the error signal representative of the residual noise power in the target zone, the controller analyzing sub-bands of the reference signal and the error signal without identification of a secondary path, and providing drive signals to the actuator to cause the actuator to deliver the actuated signals into the target zone so as to reduce the residual noise power in the target zone.

2. The control system of claim 1, wherein the reference signal and the error signal are divided into sub-bands.

3. The control system of claim 1, wherein the drive signal provided by the controller has an amplitude equal to an estimated amplitude of the noise or vibration in the target zone, and opposite in polarity to the estimated noise or vibration from the source in the target zone.

4. The control system for reducing noise in a target zone of claim 1, wherein the controller is adapted to form an adaptive filter.

5-7. (canceled)

8. A controller for reducing noise or vibration in a target zone, the noise produced by a source and transferred to the target zone by a main path, the controller comprising:
a computational system running a control algorithm, the control algorithm causing the computational system to receive a reference signal representative of noise or vibration produced by the source, and an error signal representative of the residual noise power in the target zone, the control algorithm causing the computational system to analyze sub-bands of the reference signal and the error signal without identification of a secondary path to update adaptive filter coefficients.

9-14. (canceled)

15. A control system for reducing noise or vibration in a target zone, the noise or vibration produced by a source and transferred to the target zone by a main path, the control system, comprising:
an actuator positioned to deliver actuated signals into at least a portion of the target zone;
at least one error sensor monitoring the residual noise or vibration power in the target zone and producing an error signal representative thereof;
a controller receiving a reference signal representative of noise or vibration produced by the source, and the error signal representative of the residual noise power in the target zone, the controller analyzing of the reference signal and the error signal without identification of a secondary path, and providing drive signals to the actuator to cause the actuator to deliver the actuated signals into the target zone so as to reduce at least one of a single-tone sinusoid and a multiple-frequency sinusoid in the target zone.
